(19) United States
(12) Patent Application Publication
(10) Pub. No.: US 2013/0245759 A1
(43) Pub. Date: Sep. 19, 2013

(54) MEDICAL DEVICES INCORPORATING SILICONE NANOPARTICLES, AND USES THEREOF

(71) Applicant: The Florida International University Board of Trustees, (US)

(72) Inventors: Narayanan Nair, Coral Gables, FL (US); Madhavan P.N. Nair, Coral Gables, FL (US); Sudheesh Pilakka Kanthikeel, Miami, FL (US)

(73) Assignee: THE FLORIDA INTERNATIONAL UNIVERSITY BOARD OF TRUSTEES, Miami, FL (US)

(21) Appl. No.: 13/785,143

(22) Filed: Mar. 5, 2013

Related U.S. Application Data
(60) Provisional application No. 61/608,744, filed on Mar. 9, 2012.

(51) Int. Cl. A61F 2/00; A61F 2/12

(52) U.S. Cl. CPC A61F 2/0059; A61F 2/12; B82Y 30/00
USPC 623/8; 977/779; 977/902

(57) ABSTRACT

Provided herein are uses of silicone nanoparticles as breast implant materials that reduce immunogenic responses, compared to other breast implant materials.

### CROSS-REFERENCE TO RELATED APPLICATIONS

The benefit of U.S. Provisional Application No. 61/608,744, filed Mar. 9, 2012, the disclosure of which is incorporated by reference in its entirety.

### BACKGROUND

Over the past few years, silicone based materials are being used widely in the cosmetic industry, aesthetic medicine, plastic surgery, vaccine construction, medical tools and devises. Silicone gel-containing breast implant, which was introduced over 30 years ago, has been widely used for augmentation and reconstructive mammoplasty. Current report from the national surveys have shown that more than 5% of the female population in United States has breast implants.(1) Silicone was initially believed to be biologically inert. However, silicone being a component of proteoglycans, it is possible that it may induce immunological reactions. Previous studies report that many of silicone breast implant subjects demonstrate mild immunological reactions.(2) Direct, indirect and preclinical evidences also suggest that implanted silicone induces an inflammatory reaction.(3) Previous in vivo studies in animal model have shown that liquid silicone when injected into laboratory animals elicited a mild inflammatory response in a dose-independent manner.(4, 5)

The tissue response to silicone gel breast implants typically includes an inflammatory infiltrate that consists of macrophages, lymphocytes and plasma cells suggesting that silicone implants may activate a variety of immune cells.(6) Previous studies have identified activated T cells at the biomaterial site in silicone breast implants (SBI). Studies also suggest that silicone gel or higher molecular weight silicone oils can behave as a weak immunological adjuvant or reactive biomaterial surface capable of enhancing antigen-specific immune reactions.(7-9) Animal experiments have shown that exposure of various antigens emulsified in silicone gel when introduced to mice or rats induce a greater antibody reaction than if antigen alone is given. These experimental animal data further support an association of microsilicone particles with immune effects in humans. (7-9)

Proinflammatory cytokines including IL-6 and TNF-α are secreted by monocytes/macrophages. In-vitro experiments have shown that biomaterial adherent macrophages secrete IL-1β (a soluble mediator of inflammation) and IL-6 (important for the proliferation and differentiation of B cells) in a dose dependent fashion. IL-1β, IL-6 and TNF-α as well as soluble TNF receptor were also reported to be significantly higher in women with SBI compared to age-and sex-matched controls without implants(2, 10), although no significant IL-6 differences were reported in another study with breast implants compared to controls(11). Although several clinical, preclinical and in vitro studies suggest adverse immunological effects of silicone, the ability of silicone materials to induce a robust specific immune reaction or a nonspecific inflammatory response remains to be clearly elucidated yet. Thus the controversy regarding the ability of silicone materials to induce a specific immune reaction and/or a nonspecific inflammatory response still persists.

Several clinical, preclinical and in vitro studies have reported adverse immunological effects of silicone due to their ability to induce proinflammatory molecules such as TNF-α and IL-6. In recent years, use of nanoparticles is under fast development for therapeutic drug targeting, diagnostic imaging and immune response in various field of nanomedicine. Thus, a need exists for materials that reduce the immune response.

### SUMMARY

Provided herein are medical devices that comprise silicone nanoparticles (SNP) in an implant. The SNP exhibit reduced inflammatory and immunological reactions, compared to liquid silicone and silicone microparticles. More specifically, provided herein are medical devices comprising an implant, e.g. an implant suitable for insertion under a subject (e.g., a human) skin, such as for use as breast implant, or an implant for testicles, pectorals, chin, cheek, calf, or buttock. The device comprises nanosilicone disposed within the implant. The nanosilicone can have a particle size of 100 nm or less, 70 nm or less, or 50 nm or less, or about 1 to about 100 nm, about 1 to about 80 nm, or about 1 to about 60 nm. The nanoparticles can be disposed within a container, the container defining a volume. The volume can be about 0.1 mL to about 500 mL, and selected based upon the desired end use of the device. The container can be flexible and can be closed. The container can comprise an elastomeric material, such as a silicone rubber elastomer.

### BRIEF DESCRIPTION OF THE DRAWINGS

FIG. 1 shows that the average particle size of prepared nanosilicone calculated from SEM observation was 20-100 nm.

FIG. 2 shows SMP induce production of IL6 (2A), TNF-α (2B) and IFN-γ (2C). Concentrations of IL6, TNF-α and IFN-γ in supernatants from PBMC cultured with 1, 10 & 100 μg/ml of SNP and SMP for 24 hrs were measured by ELISA. PBMC cultured in medium only served as negative control and those cultured in PHA/LPS served as positive control. Results shown are from 4 independent experiments using cells from different healthy donors without any history of using silicone devices. Concentrations are presented as mean±S.D of pg/ml. Significance is calculated with respect to negative control and between nanoslicone and microsilicone. p-value<0.05 is considered significant. The maximum stimulatory concentration of both PHA and LPS were previously determined to be 10 μg/ml and 1 μg/ml respectively. Experiments were performed at 24 hrs and 48 hrs, and since results obtained at 24 hrs showed maximum stimulatory response, this time period was selected in all other experiments.

FIG. 3 shows the effect of SNP and SMP on IL6 (3A), TNF-α (3B) and IFN-γ (3C) gene expression. PBMC were cultured with 1, 10 & 100 μg/ml of SNP and SMP for 24 hrs, RNA was extracted, reverse transcribed followed by quantitative real-time PCR for IL6, TNF-α and IFN-γ along with housekeeping gene GAPDH-specific primers. Data are expressed as mean±S.D of TAI values of 4 independent experiments using cells from different healthy donors without any history of using silicone devices. PBMC cultured in medium only served as negative control and those cultured in PHA/LPS served as positive control. Significance is reported with respect to negative control and between SNP and SMP. p-value<0.05 is considered significant.

FIG. 4 shows SMP induce intracellular expression of TNF-α (4A) and IFN-γ (4B). Cultured PBMCs were surface stained with CD3 and CD4 followed by washing with FACS buffer and permeabilization. The cells were stained with IFN-γ and TNF-α. Stained cell were acquired on flowcytometer and the percentages of intracellular IFN-γ and TNF-α-positive cells were analyzed using FlowJo software. PBMC cultured in medium only served as negative control and those cultured in PHA served as positive control. Significance is reported between SNP and SMP. p-value<0.05 is considered significant.

FIG. 5 shows the influence of different SNP and SMP on cellular viability. A viability assay using the tetrazolium salt XTT was performed 24 hrs days after treatment of PBMC with different concentrations of SNP and SMP.

FIG. 6 is a partial schematic view in cross-section illustrating a medical device or implant assembled in accordance with the teachings of the present invention and shown implanted within human tissue. The implant 20 having a container 22 and filled with nanosilicone 24 is implanted within a human breast 10 under the skin 12 and under tissue 14.

FIG. 7 shows the effect of SNPs and SMPs on cytotoxicity and apoptosis in human PBMCs.

FIG. 8 shows the response of T-cells, NK-cells, and monocytes to SNPs and SMPs.

FIG. 9 shows the response of unactivated and activated T-cells to SNPs and SMPs.

### DETAILED DESCRIPTION

The induction of immune response by silicone, specific or nonspecific, if any, can be prevented by using silicone nanoparticles (SNP) that may be unrecognizable by the immune system to induce an immune response. Thus, disclosed herein are SNP devices having improved properties, as measured by, e.g., induction of gene expression and secretion of proinflammatory molecules such as IL-6, TNF-α and IFN-γ by naïve PBMC, compared to silicone microparticles (SMP) in identical culture conditions.

Several previous in vitro, preclinical and clinical studies suggest that silicone breast implants may be associated with chronic immune activations that include dysregulation of cytokine production, natural killer cell functions, super antigen response, T cell response, autoantibody response, hypergammaglobulinemia, etc (3,16). Vojdani et al reported that 60 percent of individuals with silicone breast implants (SBI) demonstrated significantly higher T-helper/suppressor ratio compared to 10 percent in age and sex matched controls.(17) Increased T-cell stimulation indices and T-cell proliferation have also been reported in women with SBIs.(6, 18) T-cells isolated from SBI also showed increased activation marker (HLA-DR) expression and decreased T-helper cells compared to PBMCs from same donor indicating an immune activation at target tissue site, which suggests that “silicone may be immunogenic”.(16, 19) However another recent study using flow cytomerty analysis found no change in the peripheral lymphocyte subsets distribution profile in SBIs compared with normal controls although no functional studies were involved in this study(6).

In recent years, use of nanoparticles is under fast development for therapeutic drug targeting, diagnostic imaging and immune responses in various fields of nanomedicine. Recently emerging results show that differently modified silica nano particles can be of significance in various fields. For example, in nuclear medicine: (a) radioisotope labeled fluorescent silica nanoparticles are being used as detecting method for PET (positron emission tomography) and fluorescence detecting(20), (b) functionalized silica nanoparticles have promising potential as organic lymphatic tracer in biomedical imaging such as pre- and intra-operative surgical guidance, (c) use of silica nanoparticle probe in intraoperative detection and imaging of nodal metastases, differential tumor burden, and lymphatic drainage patterns in melanoma(21). Silicone NPs can also be used as a drug carrier in specific drug targeting (22) and also used to deliver insulin into Caco-2 cells(23). Use of nanoparticles has an advantage of fast absorption to the target cells and at the same time and of being prevented from immune recognition by the host immune system. Silicon-based micro materials are also being used for diagnostic and therapeutic devices for drug delivery, neural electrodes(24) and implantable sensors(25) for use within the body. Immunogenicity of material includes not only the response of immune cells to the surface, but also the cellular signals released that may initiate immune cell migration and encapsulation, or inflammation.

A recent study comparing the immunogenicity of nonporous and micropeaked silicone showed that micropeaked surface is more immunogenic than nano porous silicone(26). Several previous in vitro and animal studies have reported mild to moderate immune responses and alteration of immune cell subsets in models that closely resemble the in-vivo environment of the silicone breast implant. Tavazzani et al showed evidences of cell activation and increased IL-1 secretion by human monocyte-derived macrophages following silicone exposure, although T-cell proliferation or IL-2 secretion were not affected. The inflammatory cytokines produced by monocytes exposed to micro peaked silicone was significantly higher than normal control cultures, although nanopeaked silicone was not included in the study. Vallhov et al recently reported that mesoporus silica particles induced the production of IL-12 p70, a proinflammatory cytokine involved in both innate and adaptive immunity and upregulated the expression of CD86 by monocyte derived dendritic cells suggesting that immune regulatory signals are induced in a size and concentration dependent manner(27). The ability of certain nanomaterials to induce cytokine production appears to be dependent on a variety of factors like material composition, size, and method of delivery.

To understand the effect the SNP and SMP have in modulating expression of cytokines, we studied the ability of SNP/SMPs to modulate the production of TNF-α, IL-6 and IFN-γ; cytokines that represent critical pathways involved in the inflammatory response and differentiation processes, and are the major mediators of acute phase response playing a significant role in the pro inflammatory cascade. Previous studies have reported that women with silicon implants have silicon levels double that of control; 230±220 ng/ml vs. 130±70ng/ml (28). However the in-vitro studies are of acute studies and involved only one time treatment; whereas women with silicone breast implant studies reported in the literatures are of chronic in-vivo studies where women are exposed to silicone for a longer period although these in-vivo levels are very less compared to in-vitro acute studies used in the current experiment. In our in-vitro studies we have employed nano- and microsilicone in a comparative one time acute study under identical experimental condition, to show their immune stimulatory effect. Our studies show that level of proinflammatory cytokines produced by naïve PBMC exposed to microsilicone was significantly higher than negligible levels of IL-6, TNF-α and IFN-γ produced by PBMC exposed to nanosilicone at similar concentrations studied in identical culture conditions. Further, we have correlated the secretion of IL-6, TNF-α and IFN-γ with specific mRNA level and showed that nanosilicone did not stimulate either of these cytokine gene expression compared to higher level of IL-6, TNF-α and IFN-γ specific gene expression induced by microsilicone. These results clearly show that microsilicone and nanosilicone differentially affect the proinflammatory cytokine production by naïve PBMC which may be of clinical significance in various field of nanomedicine.

One of the primary concerns while using nano materials is that of possible toxicity. Evaluating cell viability is important for the application of silicon-based materials for potential clinical use. High amounts of non-regulated cell death, in conjunction with a foreign material, can lead to a heightened inflammatory response, resulting in a broad chronic inflammation and perhaps a nonspecific systemic attack on other cells and tissues. Whether, the inability of PBMC exposed to nanosilicone to secrete cytokines was not due to the toxicity produced by nanosilicone on target cells has been examined in XTT viability test system. The results showed that SNPs were not toxic to PBMC suggesting that non stimulatory effect of SNP is not due to the toxicity induced by SNP on PBMC.

Our results suggest that SNP fail to induce an immune reaction even at a higher concentration compared to SMP. Our finding can be of therapeutic significance in breast implant subjects who use microsilicone based devises. Replacing the micro silicone particles with silicone nanoparticles can be of significant advantage to avoid immune recognition and subsequent silicone related adverse effects in breast implants or reconstructive mammoplasty.

Thus, disclosed herein are implants suitable to be implanted into a body, e.g., the breast, testicles, pectorals, a chin, cheeks, a calf, buttocks or other parts of the human or an animal body. The implant comprises as a filler nanosilicone as disclosed herein. The nanosilicone is sterile, and pharmaceutically acceptable, e.g., suitable to be implanted into a subject (e.g., a mammal, such as a human). The implant is filled under sterile conditions.

Nanosilicone are silicone particles having an average diameter of 100 nm or less, e.g., 90 nm or less, 80 nm or less, 70 nm or less, 60 nm or less, 50 nm or less, or 40 nm or less. Microsilicone are silicone particles having an average diameter of 200 nm or greater.

The nanosilicone can be disposed within a container 10 (see FIG. 6) made of a material suitable for being implanted into a subject, e.g. a mammal, such as a human, for example. In some cases, the container is closed. The container can be flexible. The container can be impermeable to the nanosilicone, or can be minimally permeable. In cases where the container is minimally permeable, the filler can leak into the surrounding tissue of the implant. In such cases, then, the use of the nanosilicone provides a benefit over use of microsilicone or other silicone materials because it minimizes the immunological response in the subject.

Some examples of materials for use as the container include an elastomer, such as silicone rubber elastomer. The elastomer can be silicone rubber, a laminate of various forms of silicone, silicone copolymers, polyurethane, and various other elastomers in various combinations.

### Examples

### Preparation of Silicone Nanoparticles (SNP)

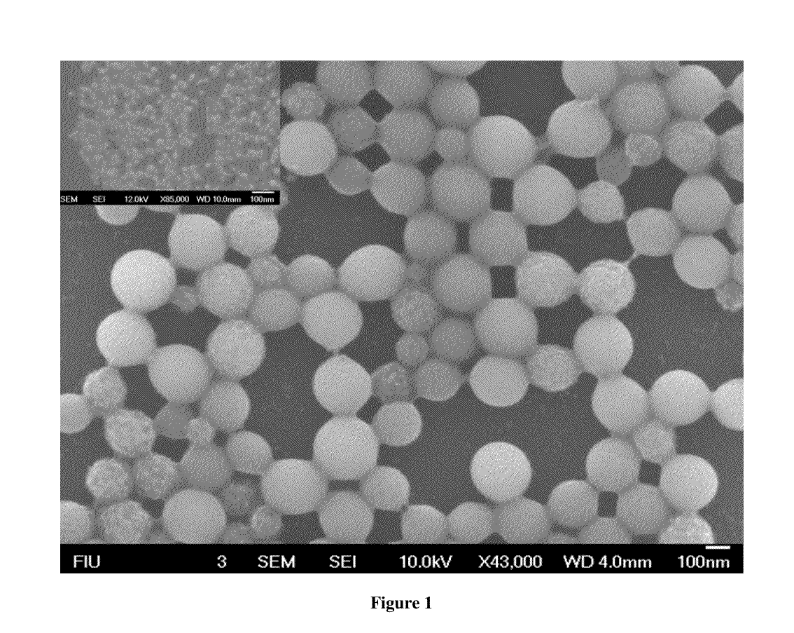

SNP was prepared by a modified method of Rao K S et al.(12) Briefly, SNPs were prepared by the co-hydrolysis of tetraethyl orthosilicate (TEOS, SiC₈H₂₀O₄; sigma) in ethanol containing ammonia at an intermediate stage of the synthesis. 0.4 M ethanol was added to 70 mM TEOS drop wise with vigorous shaking using a vortex, followed by sonication for 30 min. This was followed by slow neutralization of the mixture using 3 mM ammonium hydroxide. The mixture was sonicated for 60 minutes. The resulting SNPs were washed twice and then resuspended in PBS. Particle size and morphology of resultant particles were determined on a Phillips CM120 Transmission Electron Microscope (TEM). The size distribution of these SNPs is in the range of 20-100 nm (FIG. 1). As controls, dioxide based silicone microparticles (SMP) (2 μm) were commercially purchased (Fluka; cat no: 81108). Both SNP and SMP were used at 1, 10 and 100 μg/ml.

### Preparation of Peripheral Blood Mononuclear Cells (PBMC)

PBMCs were isolated from whole blood obtained from naïve normal subjects by standard, density gradient centrifugation as previously described.(13, 14) Briefly, leukopack was diluted by adding five volumes of phosphate-buffered saline (PBS) and overlayed over histopaque (1.077 g/ml, H889, Sigma Aldrich, St. Louis, Mo.). The samples were centrifuged at 1200×g for 20 min at room temperature. PBMCs were carefully retrieved from the interface and washed twice with PBS. PBMCs (2×10⁶ cells/ml) were cultured in RPMI-1640 media supplemented with 10% FBS and 1X Pen-Strep (Gibco, Life Technologies, Coral Island, N.Y.) with different concentrations of SNP and SMP (1, 10 and 100 μg/ml) for 24 hrs. Lipopolysacharide (LPS) (1 μg/ml) and phtohemagglutinin (PHA) (10 μg/ml) were used as positive stimulant controls as previously determined.

### ELISA

After termination of culture, supernatants were quantified for IL-6, TNF-α and IFN-γ secretion by ELISA (R& D System) as per the manufacturer's recommendation.

RNA extraction and real-time quantitative PCR: Total RNA from 2×10⁶ cultured PBMCs was extracted using the Qiagen kit (Invitrogen Life Technologies, Carlsbad, Calif., USA) following the manufacturer's instructions. The total RNA (5 μg) was used for the synthesis of the first strand of cDNA. The amplification of cDNA was performed using specific primers for IL-6, TNF-α and IFN-γ. GAPDH was used as housekeeping gene. Relative abundance of each mRNA species was assessed using brilliant Q-PCR master mix from Stratagene (Santa Clara, Calif.) using Mx3000P instrument which detects and plots the increase in fluorescence versus PCR cycle number to produce a continuous measure of PCR amplification. Relative mRNA species expression was quantitated and the mean fold change in expression of the target gene was calculated using the comparative C_{T} method (Transcript Accumulation Index, TAI=2^{-ΔΔCT}). All the results were expressed as the ratio of normalized expression of the target gene in treated cells to the normalized expression of the target gene in untreated control cells.(15)

Surface and Intra-cellular cytokine staining: At the termination of SNP and SMP treatment, cells were surface stained with CD3 and CD4 followed by washing with FACS buffer, permeabilized for 20 min at 4° C. The cells were then resuspended in permeabilizing buffer and were stained with IFN-γ and TNF-α for 30 min. Finally, the cells were washed two times with permeabilizing buffer and were resuspended in 2% paraformaldehyde. Stained cell were acquired on FACS LSRII (Becton Dickinson, San Jose, Calif.) and the percentages of intracellular IFN-γ and TNF-α-positive cells were analyzed using FlowJo software. Becton Dickinson GolgiPlug™ was added at a concentration of 1 μl for every 1 ml of cell culture during the last 4 hrs of SNP/SMP treatment.

XTT viability assay: XTT Cell viability assay was used to screen cytotoxic agents using standard photometric microplate. This assay is based on the conversion of the water-soluble XTT (2,3-bis-(2-methoxy-4-nitro-5-sulfophenyl)-2H-tetrazolium-5-carboxanilide) reagent to an orange formazan product by actively respiring cells, the amount of which is proportional to the number of living cells in the sample and can be photometrically quantified at 475 nm. At the end of the treatment, 50 μl mixture of electron coupling reagent and XTT reagent (1:50) was added to the treated cells and incubated for 4 hrs at 37° C. At the end of the incubation, absorbance was measured at 490 and the 690 nm using a plate reader. Media, no-treatment control and ‘dead’ control were used as controls. For the ‘dead control’, wells were treated with a final concentration of 0.2% triton X-100 15 minutes before the end of the drug treatment.

Statistical analysis: Data are presented as mean±SD of 3 or more independent experiments performed in triplicate and were analyzed using Graph Pad Prism version 5.0. Differences between positive stimulant treated and untreated cultures and between SNP and SMP treated cultures were analyzed by unpaired t-test, and p<0.05 was considered to be significant.

### Results

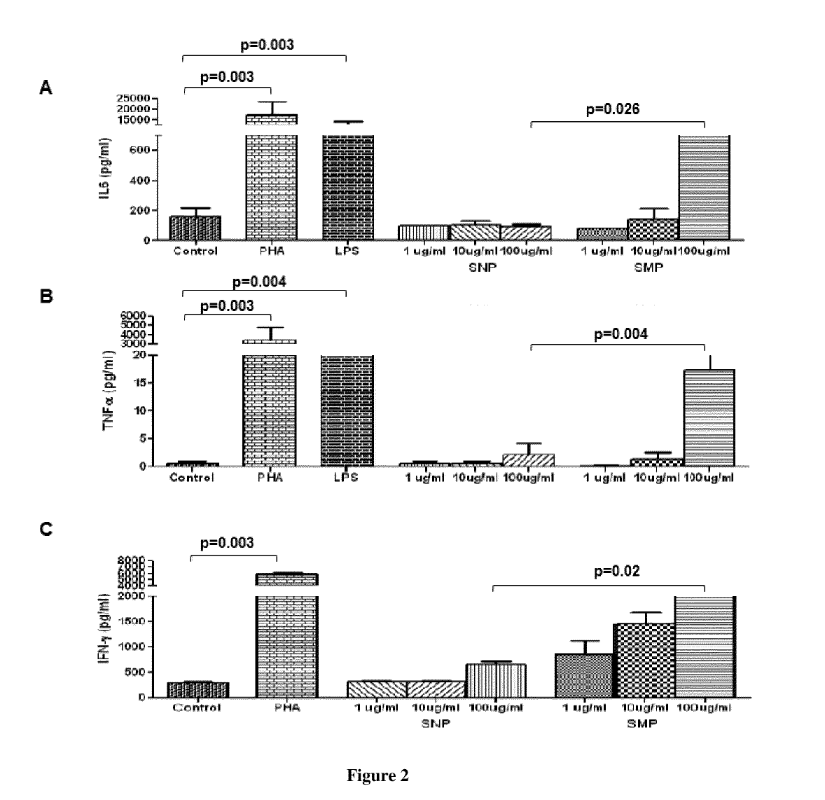

Data presented in FIG. 2A show that PBMC cultured with positive stimulants PHA and LPS produced robust induction of IL-6 (17,257 pg/ml; p=0.003 and 10,612 pg/ml; p=0.003 respectively) compared to untreated control culture (157 pg/ml). PBMC cultured with SNP at 1, 10 and 100 μg/ml did not induce significant IL-6 production and was comparable with control culture. PBMC cultured with silicone microparticles (SMP) at 1 and 10 μg/ml produced insignificant level of IL-6 (82.34 pg/ml; p=NS and 143 pg/ml; p=NS) respectively compared to similar concentrations of SNP (106.34 and 96.1 pg/ml) or negative media control cultures. However, PBMC cultured with SMP at 100 μg/ml produced significantly high level of IL-6 (1472 pg/ml; p=0.026) compared to 96 pg/ml produced by SNP at similar concentration.

Data presented in FIG. 2B show that PBMC cultured with positive stimulants PHA and LPS produced significant induction of TNF-α production (3427 pg/ml; p=0.003 and 395.6 pg/ml; p=0.004 respectively) compared to untreated control culture. Similarly, PBMC cultured with SNP at 1, 10 and 100 μg/ml did not induce significant TNF-α production and was comparable to untreated control culture. However, PBMC cultured with SMP at 100 μg/ml concentration significantly induced TNF-α production (17.3 pg/ml; p=0.004) compared to negligible level of TNF-α (2.13 pg/ml) produced by SNP at 100 μg/ml concentration.

FIG. 2C show that PBMC cultured with SNP at 1, 10 and 100 μg/ml did not induce significant IFN-γ production compared to untreated control culture. However, PBMC cultured with SMP at 100 μg/ml concentration significantly induced IFN-γ production (2023 pg/ml; p=0.02) compared to level of IFN-γ (663 pg/ml) produced by SNP at 100 μg/ml concentration. PBMC cultured with positive stimulants PHA produced significant induction of IFN-γ production (5897 pg/ml; p=0.003) compared to untreated control culture.

To study the effect of SNP on IL-6, TNF-α, and IFN-γ specific gene expression and to correlate with protein production, PBMC were cultured separately with SNP and SMP at similar concentrations and the mRNA was extracted, reverse transcribed and quantified by qPCR using specific primers against IL-6,TNF-α and IFN-γ.

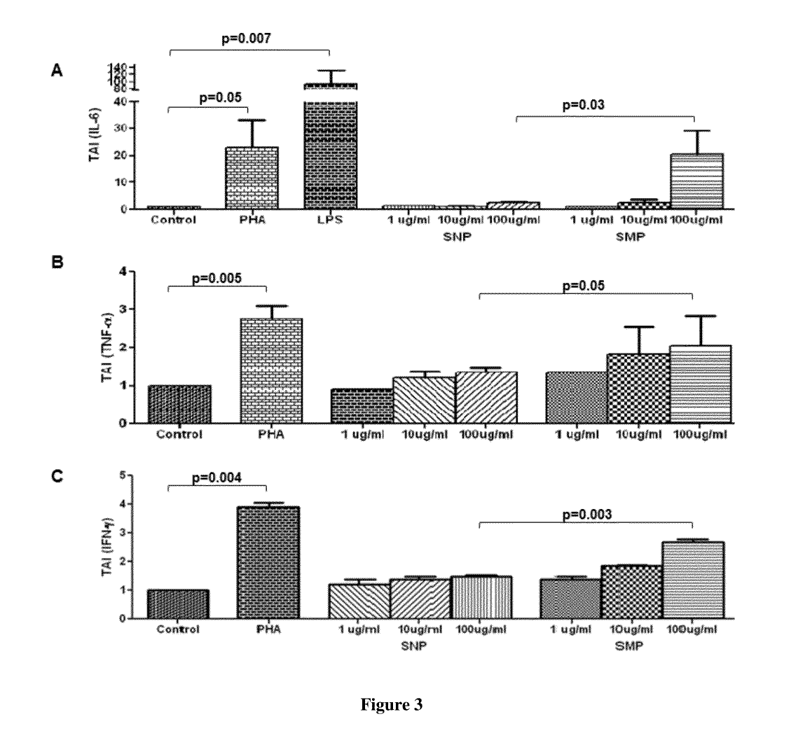

Data presented in FIG. 3A show that PBMC cultured with positive stimulants PHA and LPS produced significant induction of IL-6 gene expression (TAI=22.96; p=0.05 and 93.77; p=0.007 respectively) compared to untreated control culture (TAI=1). PBMC cultured with SMP at 100 μg/ml also induced increased level of IL-6 gene expression (TAI=20.66; p=0.03) compared to negligible induction of IL-6 gene expression induced by 100 μg/ml of SNP or negative media control cultures (TAI=1).

Data presented in FIG. 3B show that PBMC cultured with positive stimulant PHA produced significant induction of TNF-α gene expression (TAI=2.7; p=0.005) compared to untreated control culture (TAI=1). Similarly, PBMC cultured with SNP at 1,10 and 100 μg/ml also did not induce significant levels of TNF-α gene expression and was comparable to untreated control culture (TAI=1). However, PBMC cultured with SMP at 100 μg/ml concentration significantly induced TNF-α gene expression (TAI=2.06; p=0.05) compared to level of TNF-α produced by SNP at 100 μg/ml concentration. Thus the results of IL-6 and TNF-α gene expression studies complement with IL-6 and TNF-α protein production.

As shown in FIG. 3C, PBMC cultured with SNP at 1,10 and 100 μg/ml also did not induce significant levels of IFN-γ gene expression and was comparable to untreated control culture (TAI=1). However, PBMC cultured with SMP, compared to SNP, at 100 μg/ml concentration significantly induced IFN-γ gene expression (TAI=2.67; p=0.003). Positive stimulant PHA induced significant IFN-γ gene expression (TAI=3.9; p=0.004) compared to untreated control culture (TAI=1). The results of gene expression studies complement with protein production.

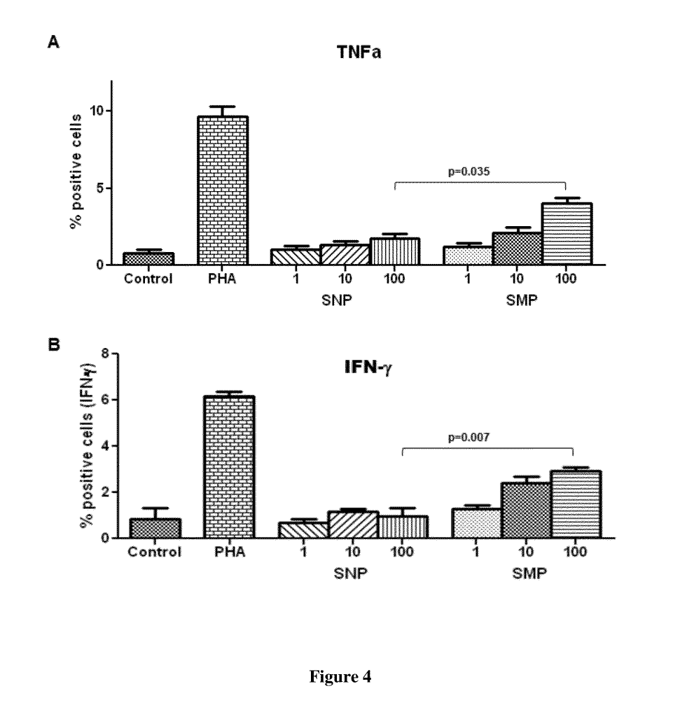

In order to reconfirm the stimulatory potential of SMP with respective to SNP, we carried out intracellular cytokine production of TNF-α and IFN-γ. PBMC cultured with SNP at 1,10 and 100 μg/ml did not produce significant intracellular TNF-α and IFN-γ production (FIG. 4A and 4B). However, PBMC cultured with SMP at 100 μg/ml concentration produced significantly high TNF-α (p=0.035) and IFN-γ (p=0.007) compared to SNP at the same concentration. In both cases, positive stimulant PHA induced significant TNF-α and IFN-γ production.

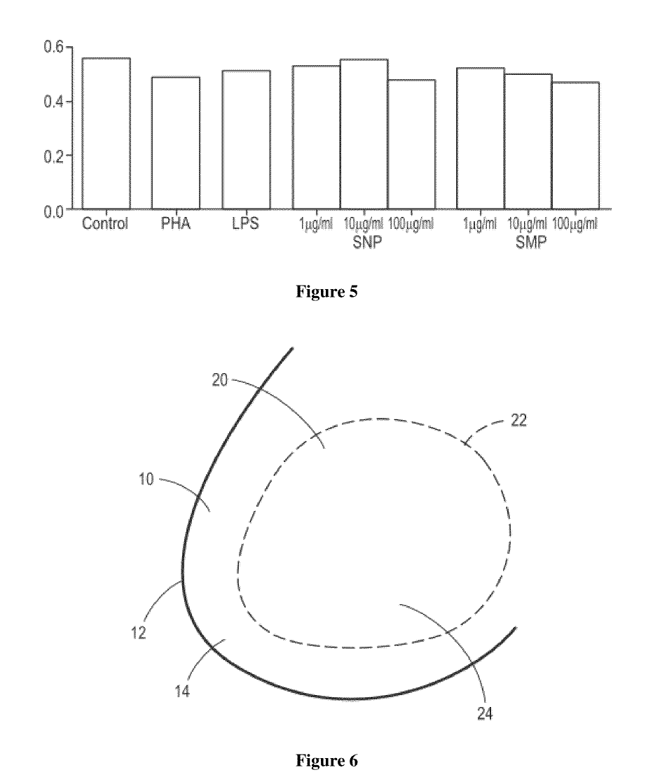

In order to examine the nonstimulatory effect of SNP by PBMC is not due to the toxicity induced by SNP, the viability of PBMC cultured with SNP and SMP was checked after 48 hrs and observed that SNP and SMP were not toxic to the PBMC as assessed by XTT viability assay (FIG. 5).

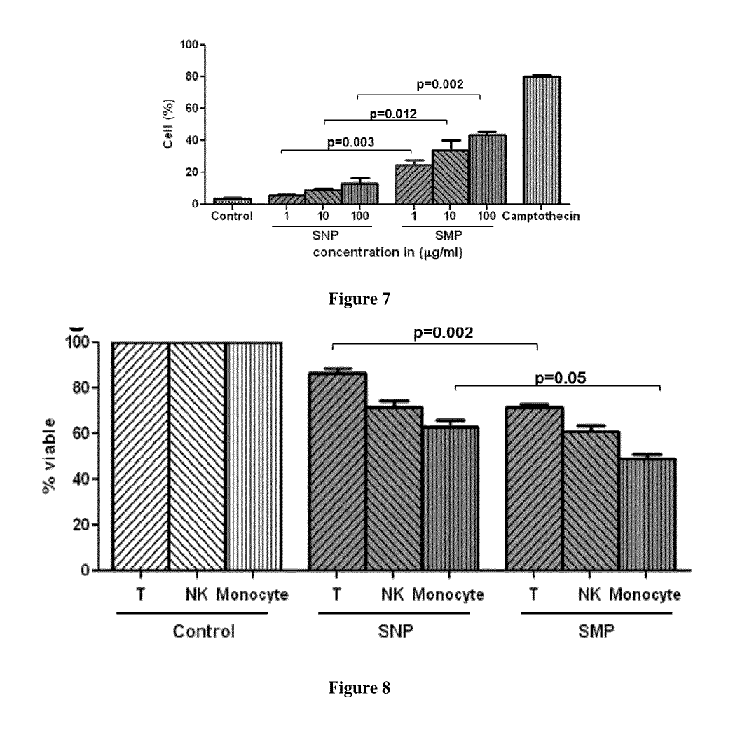

Cellular influences induced by SNP in culture medium dispersion were studied, and compared it to the effects induced by SMP. When we investigated the effect of SNP/SMPs on cytotoxicity and apoptosis in human PBMCs, we found that SMP induces apoptosis in PBMC in a dose-dependent fashion, whereas SNPs do not induce apoptosis (FIG. 7).

Since human immune system comprises of different cell types including T & B-lymphocytes, NK-cells and monocytes, we were enthusiastic to examine the response of different types of normal cells to SNP/SMP. When we stained the cells with propidium iodide (PI), a red fluorescent nuclear stain that selectively enters cells with disrupted plasma membranes, different cell types were sensitive to the particle in different degrees. T-lymphocytes (CD4-T cells) displayed the most resistance, followed by NK cells and monocytes (FIG. 8). This finding was suggestive of the fact that the different cell subtypes within the PBMC are differentially susceptible to the toxicity; i.e., toxicity was elicited in a cell-dependent manner.

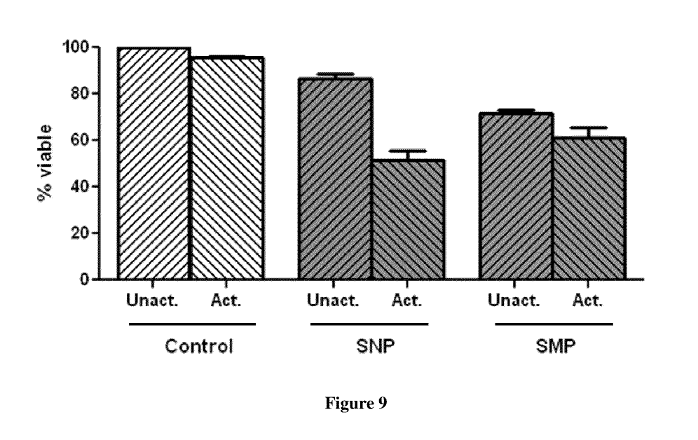

It was then investigated whether the sensitivity induced differently in resting T-cells compared to activated T-cells. Normal PBMCs were activated with stimulatory T-cell receptor (TCR) antibodies (anti-CD3) and costimulated with anti-CD28 or left untreated. TCR activated cells were more susceptible to cell death compared to unactivated resting T cells after SNP treatment (FIG. 9). These results suggest that the sensitivity of SNP/SMP to T cells is dependent on the activation state of the cell; activated cells being more susceptible to SNP than SMP. These findings may be of important clinical interest as one of the greatest challenges facing during chemotherapy for cancer is the inability of anticancer drugs to effectively distinguish between cancerous and normal cells. This indiscriminate action frequently leads to systemic toxicity and debilitating adverse effects in normal body tissues. Since SNP targets the activated cells more, binding the anticancer drugs to SNP for drug delivery in breast cancer patients with breast implants might prove beneficial.

### REFERENCES

1. Statistics., A. S. o. P. S. A. N. P. S. ASPS website.

2. Ojo-Amaize, et al. Silicone-specific blood lymphocyte response in women with silicone breast implants. Clinical and diagnostic laboratory immunology 1: 689-695, 1994.

3. Potter M, R. N. Immunology of Silicones. Heidelbergy (Germany): Spinger-Verlag Berlin, 1996.

4. Rees, et al. Visceral response to subcutaneous and intraperitoneal injections of silicone in mice. Plastic and reconstructive surgery 39: 402-410, 1967.

5. Rees, et al. An Investigation of Cutaneous Response to Dimethylpolysiloxane (Silicone Liquid) in Animals and Humans—a Preliminary Report. Plastic and reconstructive surgery 35: 131-139, 1965.

6. Prantl, et al. Flow cytometric analysis of peripheral blood lymphocyte subsets in patients with silicone breast implants. Plastic and reconstructive surgery 121: 25-30, 2008.

7. Naim, et al. Induction of type II collagen arthritis in the DA rat using silicone gels and oils as adjuvant. Journal of autoimmunity 8: 751-761, 1995.

8. Naim, et al. The effect of silicone-gel on the immune response. Journal of biomaterials science 7: 123-132, 1995.

9. Nicholson, et al. Silicone gel and octamethylcyclotetrasiloxane (D4) enhances antibody production to bovine serum albumin in mice. Journal of biomedical materials research 31: 345-353, 1996.

10. Rodriguez, et al. Quantitative in vivo cytokine analysis at synthetic biomaterial implant sites. Journal of biomedical materials research 89: 152-159, 2009.

11. Garland, et al. Multiple myeloma in women with silicone breast implants. Serum immunoglobulin and interleukin-6 studies in women at risk. Current topics in microbiology and immunology 210: 361-366, 1996.

12. Rao, et al. A novel method for synthesis of silica nanoparticles. J Colloid Interface Sci 289: 125-131, 2005.

13. Boyum, A. Isolation of mononuclear cells and granulocytes from human blood. Isolation of monuclear cells by one centrifugation, and of granulocytes by combining centrifugation and sedimentation at 1 g. Scandinavian journal of clinical and laboratory investigation 97: 77-89, 1968.

14. Gandhi, et al. Differential effects of HIV type 1 clade B and clade C Tat protein on expression of proinflammatory and antiinflammatory cytokines by primary monocytes. AIDS research and human retroviruses 25: 691-699, 2009.

15. Shively, et al. Soy and social stress affect serotonin neurotransmission in primates. Pharmacogenomics J 3: 114-121, 2003.

16. Tavazzani, et al. In vitro interaction between silicone gel and human monocyte-macrophages. Journal of biomedical materials research 72: 161-167, 2005.

17. Vojdani, et al. Immune functional impairment in patients with clinical abnormalities and silicone breast implants. Toxicology and industrial health 8: 415-429, 1992.

18. Ciapetti, et al. Assessment of viability and proliferation of in vivo silicone-primed lymphocytes after in vitro re-exposure to silicone. Journal of biomedical materials research 29: 583-590, 1995.

19. Brauber N, C. A., Vojdani A. Silicone breast implants and autoimmunity: causation, association, or myth? J Bio mater Sci Polym Ed 7: 133-145, 1995.

20. Yoon, et al. Specific targeting, cell sorting, and bioimaging with smart magnetic silica core-shell nanomaterials. Small 2(2): 209-15, 2006.

21. Benezra, et al. Multimodal silica nanoparticles are effective cancer-targeted probes in a model of human melanoma. The Journal of Clinical Investigation 121; 7: 2768-2780, 2011.

22. Lu, et al. Mesoporous Silica Nanoparticles as a Delivery System for Hydrophobic Anticancer Drugs. small 3 (8): 1341-1346, 2007.

23. Foraker, et al. Microfabricated porous silicon particles enhance paracellular delivery of insulin across intestinal Caco-2 cell monolayers. Pharmaceutical Research 20(1):110-166, 2003.

24. Martinez Santiesteban, et al. Magnetic resonance compatibility of multichannel silicon microelectrode systems for neural recording and stimulation: design criteria, tests, and recommendations. IEEE transactions on bio-medical engineering 53: 547-558, 2006.

25. Muthusubramaniam, et al. Hemocompatibility of silicon-based substrates for biomedical implant applications. Annals of biomedical engineering 39: 1296-1305, 2011.

26. Ainslie, et al. In vitro immunogenicity of silicon-based micro- and nanostructured surfaces. ACS Nano 2: 1076-1084, 2008.

27. Vallhov, et al. Mesoporous silica particles induce size dependent effects on human dendritic cells. Nano Lett 7: 3576-3582, 2007.

28. Tueber, et al. Elevated serum silicon levels in women with silicone gel breast implants. Biological Trace Element Research 48: 121-130, 1995.

1. A medical device comprising:
an implant; and
nanosilicone disposed within the implant.

2. The device of claim 1, wherein the nanosilicone has a particle size of 100 nm or less.

3. The device of claim 2, wherein the particle size is 70 nm or less.

4. The device of claim 3, wherein the particle size is 50 nm or less.

5. The device of claim 1, wherein the nanosilicone is disposed within a container, the container defining a volume.

6. The device of claim 5, wherein the container is flexible.

7. The device of claim 5, wherein the container is closed.

8. The device of claim 1, wherein the implant is suitable for implantation under the skin of a subject.

9. The device of claim 1, wherein the implant is sterile.

10. The device of claim 1 having a volume of 0.1 to 500 mL.

11. The device of claim 1, wherein the implant is suitable as a breast implant.

12. The device of claim 1, wherein the implant is suitable as an implant for testicles, pectorals, chin, cheek, calf, or buttock.

13. The device of claim 1, wherein the container is made of an elastomeric material.

14. The device of claim 13, wherein the elastomer is a silicone rubber elastomer.
